(19) United States
(12) Patent Application Publication
(10) Pub. No.: US 2023/0166078 A1
(43) Pub. Date: Jun. 1, 2023

(54) Extension Leg Securement System

(71) Applicant: Bard Access Systems, Inc., Salt Lake City, UT (US)

(72) Inventors: Liam Doctor, Salt Lake City, UT (US); Joshua D. Sherwood, Cottonwood Heights, UT (US)

(21) Appl. No.: 18/072,286

(22) Filed: Nov. 30, 2022

Related U.S. Application Data
(60) Provisional application No. 63/285,033, filed on Dec. 1, 2021.

(51) Int. Cl. A61M 25/00

(52) U.S. Cl. CPC A61M 25/0097; A61M 25/003

(57) ABSTRACT

An assembly including a catheter and an extension leg securement apparatus. The catheter includes a hub distally coupled to a catheter tube having two or more lumens. The hub is proximally coupled to two or more extension legs with each extension leg in fluid communication with one lumen. The extension leg securement apparatus includes an extension leg organizer configured to be detachably coupled to the two or more extension legs. The extension leg organizer can include a wire aperture and two or more channels arranged in a channel configuration, with each channel configured to receive one extension leg.

### PRIORITY

This application claims the benefit of priority to U.S. Provisional Application No. 63/285,033, filed Dec. 1, 2021, which is incorporated by reference in its entirety into this application.

### BACKGROUND

An insertion site for a catheter can become crowded with extension legs and associated connectors. For example, a common insertion site for inserting a central venous catheter (CVC) can be the neck. The insertion site during placement of the CVC can become crowded, with extension legs and associated connectors potentially obscuring the face of the patient or the insertion site. Any movement of the patient during placement of a catheter can lead to the catheter moving or being dislodged during placement. It would be beneficial to the patient and the clinician to have a securement system that organizes and secures the extension legs during placement, leading to increased comfort for the patient during use of the catheter and a more accurate placement of the catheter. Disclosed herein is an extension leg securement system and method of use that address the foregoing.

### SUMMARY

Disclosed herein is a catheter assembly that, according to some embodiments, includes (i) a catheter having two or more extension legs, where each extension leg is in fluid communication with at least one lumen extending along a catheter tube; and an extension leg securement apparatus attached to each of the two or more extension legs, where the extension leg securement apparatus includes an extension leg organizer having two or more channels configured to individually receive the two or more extension legs therein.

In some embodiments, each channel is coupled with one of the two or more extension legs via a press fit, a snap fit, or an interference fit. In some embodiments, each channel is configured to inhibit longitudinal displacement of the one of the two or more extension legs coupled therewith.

In some embodiments, the extension leg organizer is configured to (i) be bent by a clinician during use into a curved shape and (ii) maintain the curved shape thereafter.

In some embodiments, the extension leg securement apparatus further includes an extension leg ring coupled with the extension leg organizer, where the extension leg ring extends around the two or more extension legs as a group. In some embodiments, the extension leg ring is disposed distal the extension leg organizer.

In some embodiments, the extension leg ring includes a slot configured to individually receive the two or more extension legs therethrough and in some embodiments, the slot defines a width that is less than a diameter of the each of the two or more extension legs.

In some embodiments, the extension leg ring includes a first arm extending along a first partial circumference of the extension leg ring and a second arm extending along a second partial circumference disposed opposite the first partial circumference, and at least one of the first arm or the second arm is deflectable so as to increase the width of the slot.

In some embodiments, the extension leg securement apparatus further includes a bendable wire coupled between the extension leg ring and the extension leg organizer and in some embodiments, the bendable wire is configured to (i) be bent by a clinician during use to define a position and an orientation of the extension leg organizer with respect to the extension leg ring, and (ii) maintain the position and the orientation of the extension leg organizer with respect to the extension leg ring.

In some embodiments, the extension leg securement apparatus further includes a second extension leg organizer coupled with the extension leg organizer, where the second extension leg organizer has two or more channels configured to individually receive the two or more extension legs therein. In some embodiments, the second extension leg organizer is disposed distal the extension leg organizer.

In some embodiments, the extension leg securement apparatus further includes a bendable wire coupled between the second extension leg organizer and the extension leg organizer, and in some embodiments, the bendable wire is configured to (i) be bent by a clinician during use to define a position and an orientation of the extension leg organizer with respect to the second extension leg organizer, and (ii) maintain the position and the orientation of the extension leg organizer with respect to the second extension leg organizer.

Also disclosed herein is a catheter extension leg securement apparatus that, according to some embodiments, includes an extension leg organizer having two or more channels configured to individually receive two or more extension legs of a catheter therein.

In some embodiments of the apparatus, each channel is configured to couple with one of the two or more extension legs via a press fit, a snap fit, or an interference fit, and in some embodiments of the apparatus, each channel is configured to inhibit longitudinal displacement of the one of the two or more extension legs coupled therewith.

In some embodiments of the apparatus, the extension leg organizer is configured to (i) be bent by a clinician during use into a curved shape and (ii) maintain the curved shape thereafter.

In some embodiments, the apparatus further includes an extension leg ring coupled with the extension leg organizer, where the extension leg ring is configured to extend around the two or more extension legs as a group.

In some embodiments of the apparatus, the extension leg ring includes a slot configured to individually receive the two or more extension legs therethrough, and in some embodiments the slot defines a width that is less than a diameter of each of the two or more extension legs.

In some embodiments of the apparatus, the extension leg ring includes a first arm extending along a first partial circumference of the extension leg ring and a second arm extending along a second partial circumference of the extension leg ring, where the second partial circumference is disposed opposite the first partial circumference, and where at least one of the first arm or the second arm is deflectable so as to increase the width of the slot.

In some embodiments, the apparatus further includes a bendable wire coupled between the extension leg ring and the extension leg organizer, and in some embodiments, the bendable wire is configured to (i) be bent by a clinician during use to define a position and an orientation of the extension leg organizer with respect to the extension leg ring, and (ii) maintain the position and the orientation of the extension leg organizer with respect to the extension leg ring.

In some embodiments, the apparatus further includes a second extension leg organizer coupled with the extension leg organizer, where the second extension leg organizer has two or more channels configured to individually receive the two or more extension legs therein.

In some embodiments, the apparatus further includes a bendable wire coupled between the second extension leg organizer and the extension leg organizer, and in some embodiments, the bendable wire is configured to (i) be bent by a clinician during use to define a position and an orientation of the extension leg organizer with respect to the second extension leg organizer and (ii) maintain the position and the orientation of the extension leg organizer with respect to the second extension leg organizer.

Also disclosed herein is a method of using a catheter that, according to some embodiments, includes (i) coupling two or more extension legs of the catheter with an extension leg organizer, where the extension leg organizer includes two or more channels configured to receive the two or more extension legs therein; and (ii) disposing the two or more extension legs within an extension leg ring coupled with the extension leg organizer.

In some embodiments of the method, the extension leg organizer is coupled with the extension leg ring via a bendable wire, and the method further includes bending the bendable wire to define a position and an orientation of the extension leg organizer with respect to the extension leg ring.

In some embodiments of the method, the extension leg ring includes a slot configured to individually receive the two or more extension legs therethrough, where the slot defines a width that is less than a diameter of each of the two or more extension legs, and disposing the two or more extension legs within an extension leg ring includes displacing the two or more extension legs through the slot.

In some embodiments of the method, the extension leg ring includes a first arm extending along a first partial circumference of the extension leg ring and a second arm extending along a second partial circumference of the extension leg ring, where second partial circumference is disposed opposite the first partial circumference, where at least one of the first arm or the second arm is deflectable so as to increase the width of the slot, and where displacing the two or more extension legs through the slot includes deflecting the at least one of the first arm or the second arm.

These and other features of the concepts provided herein will become more apparent to those of skill in the art in view of the accompanying drawings and following description, which describe particular embodiments of such concepts in greater detail.

### DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1 illustrates a perspective view of a catheter assembly including an extension leg securement apparatus, in accordance with some embodiments;

FIG. 2A illustrates a perspective view of the extension leg securement apparatus coupled with extension legs of a catheter, in accordance with some embodiments;

FIG. 2B is a detailed end view of the extension leg organizer of FIG. 1, in accordance with some embodiments;

FIG. 2C is a detailed end view of another embodiment of the extension leg securement organizer, in accordance with some embodiments;

FIG. 2D is a detailed end view of the extension leg ring of FIG. 1, in accordance with some embodiments;

FIG. 2E illustrates a cross-sectional exploded side view of the extension leg securement apparatus, in accordance with some embodiments;

FIG. 3A illustrates a perspective view of another embodiment of the extension leg securement apparatus including a pair of extension leg organizers of FIG. 2C, in accordance with some embodiments;

FIG. 3B illustrates a perspective view of another embodiment of the extension leg securement apparatus including a pair of extension leg organizers of FIG. 2B, in accordance with some embodiments;

FIG. 4A illustrates a perspective view of another embodiment of the extension leg securement apparatus in a non-bended configuration, in accordance with some embodiments;

FIG. 4B illustrates a perspective view of the extension leg securement apparatus of FIG. 4A in a bended configuration, in accordance with some embodiments; and

FIGS. 5A-5D are various illustrations of the catheter assembly of FIG. 1 depicting an exemplary method of using the extension leg securement apparatus, in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal-end portion” of, for example, a catheter disclosed herein includes a portion of the catheter intended to be near a clinician when the catheter is used on a patient. Likewise, a “proximal length” of, for example, the catheter includes a length of the catheter intended to be near the clinician when the catheter is used on the patient. A “proximal end” of, for example, the catheter includes an end of the catheter intended to be near the clinician when the catheter is used on the patient. The proximal portion, the proximal-end portion, or the proximal length of the catheter can include the proximal end of the catheter; however, the proximal portion, the proximal-end portion, or the proximal length of the catheter need not include the proximal end of the catheter. That is, unless context suggests otherwise, the proximal portion, the proximal-end portion, or the proximal length of the catheter is not a terminal portion or terminal length of the catheter.

With respect to “distal,” a “distal portion” or a “distal-end portion” of, for example, a catheter disclosed herein includes a portion of the catheter intended to be near or in a patient when the catheter is used on the patient. Likewise, a “distal length” of, for example, the catheter includes a length of the catheter intended to be near or in the patient when the catheter is used on the patient. A “distal end” of, for example, the catheter includes an end of the catheter intended to be near or in the patient when the catheter is used on the patient. The distal portion, the distal-end portion, or the distal length of the catheter can include the distal end of the catheter; however, the distal portion, the distal-end portion, or the distal length of the catheter need not include the distal end of the catheter. That is, unless context suggests otherwise, the distal portion, the distal-end portion, or the distal length of the catheter is not a terminal portion or terminal length of the catheter.

The phrases “connected to,” “coupled with,” and “in communication with” refer to any form of interaction between two or more entities, including but not limited to mechanical, electrical, magnetic, electromagnetic, fluid, and thermal interaction. Two components may be coupled with each other even though they are not in direct contact with each other. For example, two components may be coupled with each other through an intermediate component.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

Unless defined otherwise, all technical and scientific terms used herein have the same meaning as commonly understood by those of ordinary skill in the art.

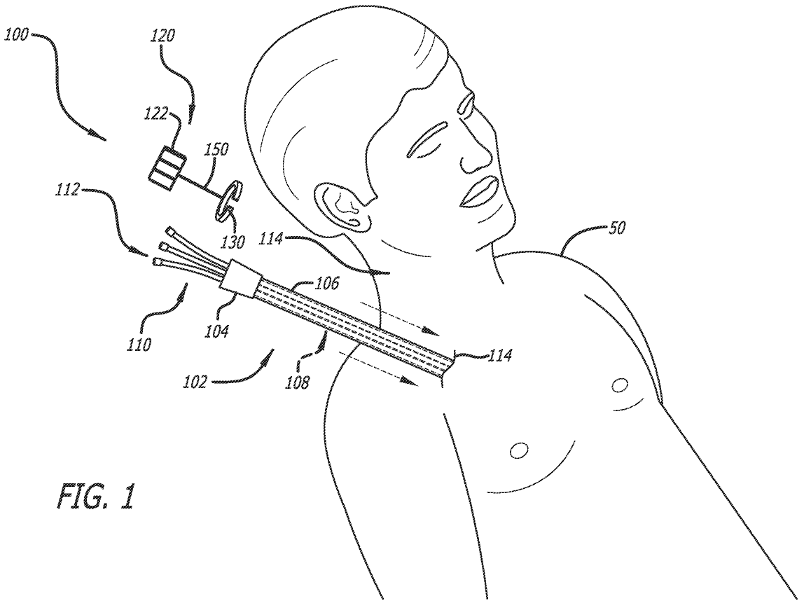

FIG. 1 illustrates a perspective view of a catheter assembly 100 including an extension leg securement apparatus 120 and a catheter 102 having a number (e.g., 2, 3, 4 or more) of extension legs 110, in accordance with some embodiments. The extension leg securement apparatus (apparatus) 120 is generally configured to organize the extension legs 110 of the catheter 102 during use of the catheter 102. In some embodiments, the apparatus 120 is configured to define a direction or orientation of the extension legs 110. In some embodiments, the apparatus 120 is configured to define an arrangement of the extension legs 110, i.e., a position of each extension leg with respect to the other extension legs.

In some embodiments, the catheter 102 may include a central venous catheter (“CVC”), although it can be appreciated that other multi-lumen catheters (e.g., peripherally inserted central catheter (“PICC”), central venous catheter (“CVC”), midline catheter, or the like) may be used under the scope of this disclosure. In some embodiments, the catheter 102 may include a hub 104 coupled between a catheter tube 106 having a number (e.g., 1, 2, 3, 4 or more) of lumens 108 therein and the extension legs 110. Each extension leg 110 is in fluid communication with at least one of the lumens 108. In some embodiments, the extension legs 110 may each include a connector 112. In some embodiments, the connector 112 may include Luer locking connector. In some embodiments, the catheter assembly 100 includes the apparatus 120 coupled with the catheter 102 to organize the extension legs 110 and/or secure the extension legs 110 together as will be described in more detail herein. In some embodiments, the apparatus 120 may be configured to detachably couple to the extension legs 110.

In the illustrated embodiment, the apparatus 120 may generally include an extension leg organizer 122 coupled with an extension leg ring 130 via a bendable wire 150. In some embodiments, either one of the extension leg organizer 122 or the extension leg ring 130 may be omitted. In some embodiments, the apparatus 120 may include more than one extension leg organizer 122 and/or more than one extension leg ring 130.

In accordance with placement of the catheter 102, an insertion site 114 on a patient 50 may be identified and accessed, the insertion site 114 may be used to access the vasculature of the patient. In some embodiments, the insertion site 114 may be in the subclavian region of the patient. The catheter 102 may be slidably inserted through the insertion site 114 into the vasculature. The extension legs 110 may be organized and secured using the apparatus 120 before, after, or during insertion of the catheter 102. In some embodiments, organizing and securing the extension legs 110 using the apparatus 120 may reduce impediments around the insertion site, thereby providing for increased patient comfort and improved catheter placement by reducing movement of the catheter 102 via reduced movement of the extension legs 110 during and/or after placement of the catheter 102. In some instances, after the catheter 102 is properly placed, the clinician or the patient may employ the apparatus 120 to keep the extension legs 110 organized. Employment of the apparatus 120 may remove the need for applying tape to the patient or other methods of organizing and/or securing the extension legs 110. In some embodiments, the apparatus 120 may be configured for single use. In other embodiments, the apparatus 120 may be configured for re-use.

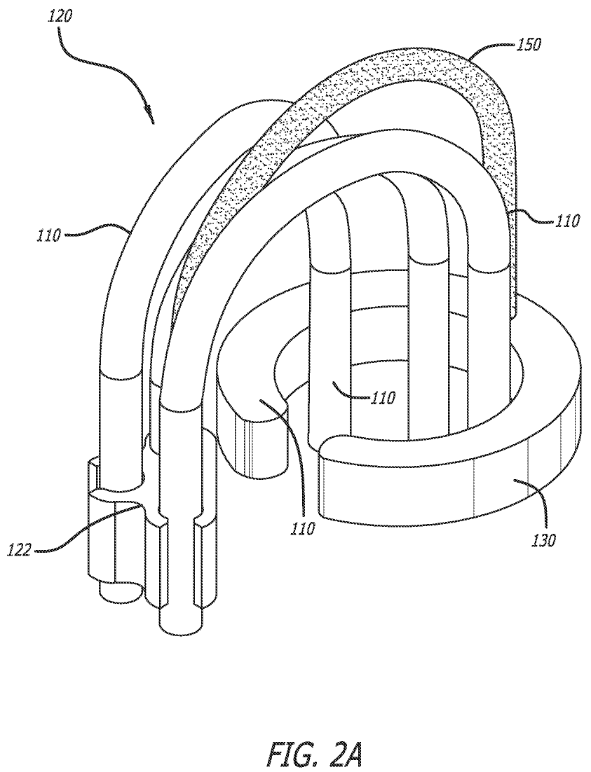

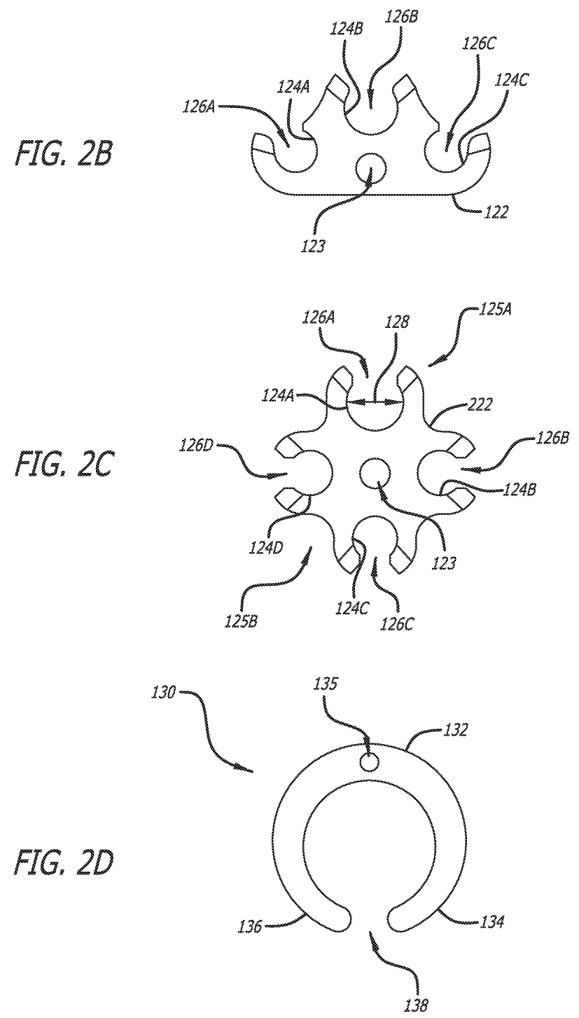

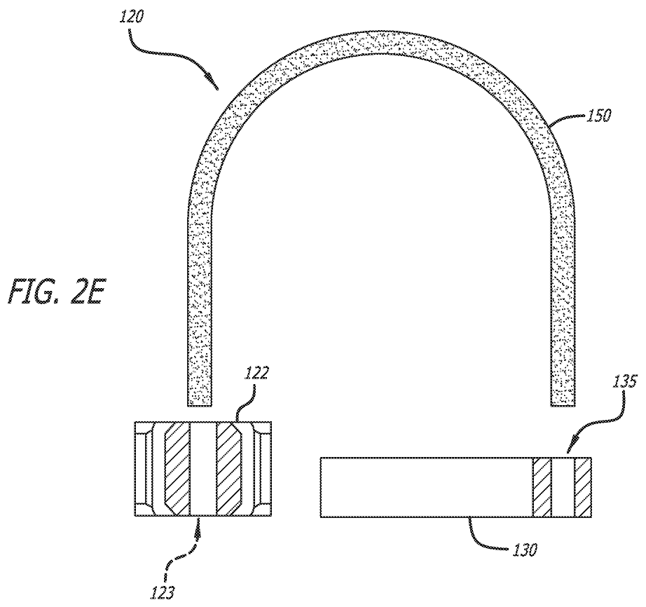

FIG. 2A illustrates a perspective view of the apparatus 120 including the extension leg organizer 122 coupled with the extension leg ring 130 via the bendable wire 150, in accordance with some embodiments. The extension leg organizer 122 may be configured to individually secure each of the extension legs 110 thereto and the extension leg ring 130 may be configured extend around or otherwise organize the extension legs 110 as a group. In some embodiments, the bendable wire 150 may be configured to be bent into any shape consistent with a desired position and/or orientation of the extension legs 110. For example, in the FIG. 2A, the bendable wire 150 is bent defining a 180 degree curve so that the extension legs 110 follow the 180 degree curve. In some embodiments, apparatus 120 may be oriented with respect to the catheter 102 such that the extension leg ring 130 is positioned distal the extension leg organizer 122. In some instances, the extension leg organizer 122 may be deployed without deploying the extension leg ring 130 or vice versa.

FIG. 2B illustrates an end view of the extension leg organizer 122, in accordance with some embodiments. In some embodiments, the extension leg organizer 122 may include a number (e.g., 2, 3, 4 or more) channels, such as the three channels 124A-124C in the illustrated embodiment, where each of the channels 124A-124C is configured to secure one extension leg 110 therein. Each of the channels 124A-124C may include a channel opening such as the corresponding channel openings 126A-126C, configured to receive the extension leg 110 therethrough. In some embodiments, the channels 124A-124C may be arranged in various channel configurations on the extension leg organizer 122, so as to present the extension legs 110 to a user so that each extension leg 110 may be easily accessed and identified.

In some embodiments, the three channels 124A-124C may be arranged in a channel configuration having a central channel 124B with each remaining channel 124A, 124C disposed laterally adjacent to the central channel 124B. This configuration allows the extension leg 110 located in the central channel 124B to be separated and easily accessible by the user. In some embodiments, the channel openings 126A-126C may be facing the same direction as illustrated in FIG. 2B, or in different directions. In some embodiments, the channel openings 126A-126C may be equal. In other embodiments, the two or more of the channel openings 126A-126C may be unequal. In some embodiments, the leg extension organizer 122 may include a wire aperture 123 configured to receive therein and couple with the bendable wire 150.

In some embodiments, each of the channels 124A-124C may include a channel diameter 128 that is greater than or equal to the channel openings 126A-126C. In some embodiments, the channel diameter 128 may be greater than a diameter of the extension legs 110 so that longitudinal displacement of the extension legs 110 with respect to the leg extension organizer 122 is freely allowed. In some embodiments, the friction between each channel each channel 124A-124C and each extension leg 110 may be configured to prevent longitudinal displacement of the extension leg organizer 122 with respect to the leg extension organizer 122. In some embodiments, with the channel openings 126A-126C may less than the diameter of the extension legs 110 so as to define a snap fit, a press fit, an interference fit, or the like.

FIG. 2C illustrates another embodiment of the extension leg organizer 222 configured to couple with and organize four extension legs 110. As such, the extension leg organizer 222 includes the four channels 124A-124D. In some embodiments, the channel configuration of the extension leg organizer 222 may be symmetrical such that the channels 124A-124D are disposed evenly along a circumference of the extension leg organizer 222 as illustrated in FIG. 2C. In other embodiments, the channels 124A-124D may be disposed unevenly along a circumference of the extension leg organizer 222, i.e., the channel configuration may be asymmetrical. In some embodiments, the number of the channels 124A-124D may correspond to the number of extension legs 110 of the catheter 102.

In some embodiments, the channel diameter 128 for each of the channels 124A-124D may be the same or the channel diameter 128 may be different for each channel 124A-124D. In some embodiments, the extension leg organizer 122 may be configured to have the four channels 124A-124D wherein the four channels 124A-124D may have a channel configuration of the four channels 124A-124D being organized into a first pair 125A of channels 124A, 124C and a second pair 125B of channels 124B, 124D. In some embodiments, the channel openings 126A, 126C of the first pair 125A may be in opposing directions and the channel openings 126B, 126D of the second pair 125B may be in opposing directions, as illustrated in FIG. 2B. In some embodiments, the first pair 125A of channels 124A, 124C may be adjacent to the second pair 125B of channels 124B, 124D, wherein each channel opening 126A, 126C of the first pair 125A of channels 124A, 124C is arranged to be 90° from each channel opening 126B, 126D of the second pair 125B of channels 124B, 124D. In some embodiments, the extension leg organizer 122 may be extruded, 3D printed, injection molded, pressed or stamped out of a sheet of material, or the like. In some embodiments, the extension leg organizer 122 may be constructed of a polymer, an elastomer, or a metal, such as aluminum, stainless steel or the like.

FIG. 2D illustrates an end view of the extension leg ring 130. In some embodiments, the extension leg ring 130 may be used with the extension leg organizer 122 to gather the extension legs 110 together in a close proximity. In some embodiments, the extension leg ring 130 may include an extension leg ring body 132 having a first arm 134 defining a first partial circumference of the extension leg ring 130 and a second arm 136 defining a second partial circumference of the extension leg ring 130, where the second arm 136 is disposed opposite the first arm 134. A slot 138 disposed between the first and second arms 134, 136 is configured to receive individually therethrough the extension legs 110 where the slot 138 defines a width that is less than a diameter of each extension leg 110 so that each extension leg 110 is inhibited from freely passing through the slot 138. One or both arms 134, 136 may be deflectable to allow for the passage of the extension legs 110 in response to a deliberation action of the clinician. In some embodiments, the extension leg ring 130 may include a wire aperture 135 configured to receive and couple with the bendable wire 150. Similar to the extension leg organizer 122, the extension leg ring 130 may be extruded, 3D printed, injection molded, pressed or stamped out of a sheet of material, or the like. In some embodiments, the extension leg organizer 122 may be constructed of a polymer, an elastomer, or a metal, such as aluminum, stainless steel or the like.

FIG. 2E illustrates a cross-sectional exploded side view of the apparatus 120 including the extension leg organizer 122, the extension leg ring 130, and bendable wire 150, in accordance with some embodiments. As discussed above, extension leg ring 130 may be coupled with the extension leg organizer 122 via the bendable wire 150. During manufacturing the of the apparatus 120, opposite ends of the bendable wire 150 may be inserted into the wire aperture 123 of the extension leg organizer 122 and the wire aperture 135 of the extension leg ring 130.

The bendable wire 150 is configured to maintain a desired shape after being bent by the clinician. In some embodiments, portions of the bendable wire 150 may be more bendable than other portions of the bendable wire 150. For example, the end portions of the bendable wire 150 coupled to the wire aperture 123 of the extension leg organizer 122 and the wire aperture 135 of the extension leg ring 130 may be more rigid (i.e., less bendable) than a central portion of the bendable wire 150 extending between the ends portions. In some embodiments, the bendable wire 150 may be pre-bent into a specific shape, such as the 180 degree shape illustrated in FIG. 2E or any other shape.

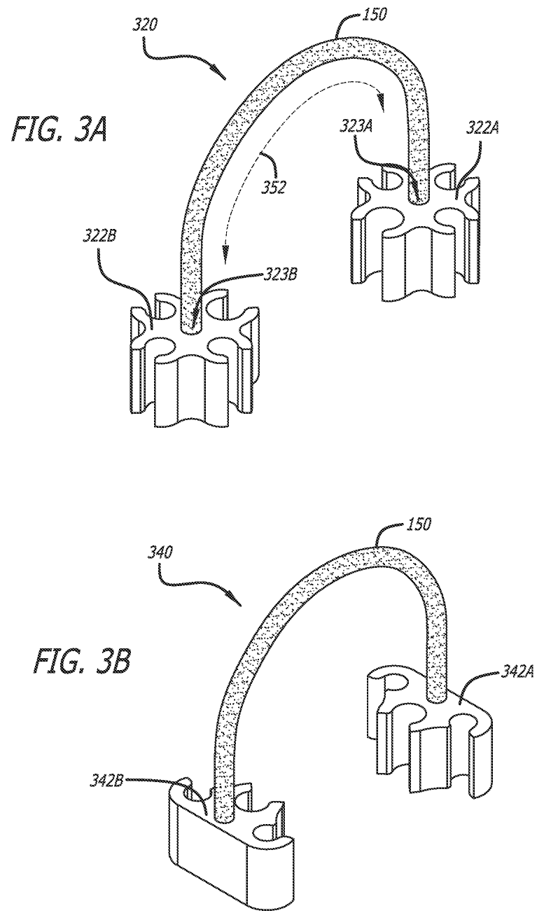

FIG. 3A illustrates a perspective view of another embodiment of the extension leg securement apparatus. The extension leg securement apparatus 320 includes a first extension leg organizer 322A and a second extension leg organizer 322B coupled with each other via the bendable wire 150, where each of the extension leg organizers 322A, 322B may, in certain respects, resemble the extension leg organizer 222 of FIG. 2C. In some embodiments, the pair of extension leg organizers 322A, 322B may more firmly organize and secure the extension legs 110 than the extension leg securement apparatus 120. In some embodiments, the first extension leg organizer 322A may have a similar channel configuration to the second extension leg organizer 322B or the first extension leg organizer 322A may have a different channel configuration to the second extension leg organizer 322B. In some embodiments, the extension leg securement apparatus 320 may come pre-assembled or the extension leg securement apparatus 320 may include the individual components (e.g., the extension leg organizers 322A, 322B, and the bendable wire 150) disassembled and so that the clinician may assemble the extension leg securement apparatus 320 in a desired configuration at the point of use. In an embodiment, where the extension leg securement apparatus 320 is pre-assembled, the bendable wire 150 may define a first length 352 separating the first extension leg organizer 322A from the second extension leg organizer 322B. In an embodiment, where the extension leg securement apparatus 320 has the individual components disassembled, the user may decide the first length 352 of the bendable wire 150 and the channel configuration of each of the first extension leg organizer 322A and the second extension leg organizer 322B. In some embodiments, the bendable wire 150 may be detachably coupled to the wire aperture 323A of the first extension leg organizer 322A and to the wire aperture 323B of the second extension leg organizer 322B. In some embodiments, the bendable wire 150 may be detachably coupled to each of the wire apertures 323A, 323B of the extension leg organizers 322A, 322B in a press fit, snap fit, interference fit, or the like.

FIG. 3B illustrates a perspective view of an embodiment of the apparatus 340 including the extension leg organizer 342 , where each of the extension leg organizers 342A, 342B may, in certain respects, resemble the extension leg organizer 122 of FIG. 2B. In some embodiments, the first extension leg organizer 342A may be oriented in the same direction as the second extension leg organizer 342B. In some embodiments, having the first extension leg organizer 342A in the same direction as the second extension leg organizer 342B allows the extension legs 110 to remain in the same configuration when the bendable wire 150 is bent. In some embodiments, the bendable wire 150 allows the extension legs 110 to follow the bend of the bendable wire 150, which may reduce stress the extension legs 110 and/or the catheter tube 106. Furthermore, the first extension leg organizer 342A and the second extension leg organizer 342B provide a simplified interface for a clinician to readily access each extension leg 110 by removing the desired extension leg 110 from either of the extension leg organizers 342A, 342B.

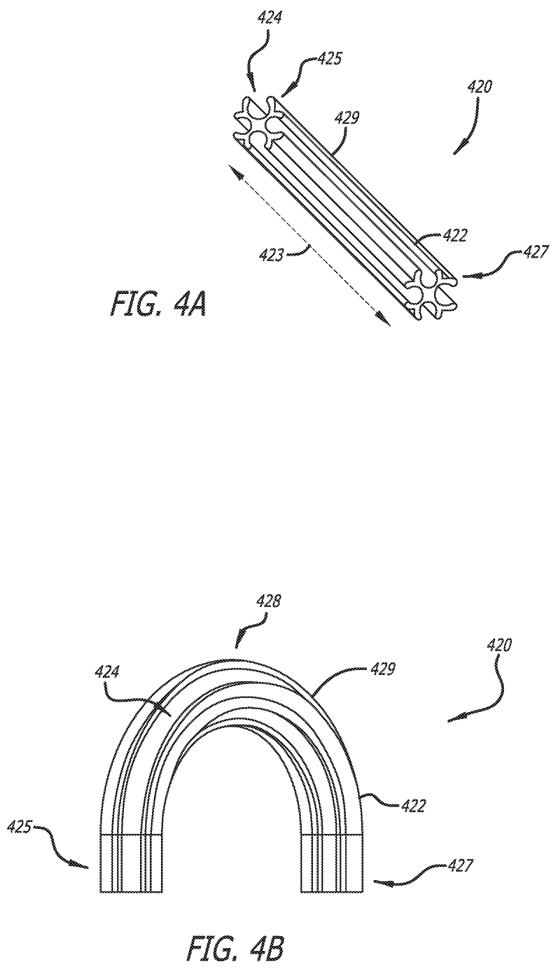

FIG. 4A illustrates a perspective view of another embodiment of the extension leg securement apparatus. The extension leg securement apparatus 420 includes of an elongated extension leg organizer 422, in accordance with some embodiments. The extension leg organizer 422 may have an elongate body 429 that extends along a length 423. The elongate body 429 extends between a proximal end 425 and a distal end 427. The elongated extension leg organizer 422 includes a number (e.g., 2, 3, 4, or more) of channels 424. In some embodiments, each channel 424 may extend along the first length 423. In some embodiments, the extension leg organizer 422 may be constructed of one or more materials that are flexible, allowing the entire extension leg organizer 222 to transition between a non-bended orientation and a bended orientation.

FIG. 4B illustrates a perspective view of the extension leg securement apparatus 420 in a bended configuration, in accordance with some embodiments. In some embodiments, the extension leg organizer 422 may be disposed in a bended orientation. In some embodiments, when disposed in the bended orientation, one or both of the proximal end 425 and the distal end 427 may be bent away from the first midpoint 428. In some embodiments, the materials that construct the extension leg organizer 422 may be configured to allow the extension leg organizer 422 to remain in the bended orientation until the user transitions the extension leg organizer 422 to the non-bended orientation. In some embodiments, a material at the midpoint 428 may be different from a material at the proximal end 425 and/or the distal end 427. For example, the material at the proximal end 425 and/or distal end 427 may be more rigid than the material at the midpoint 428. In other embodiments, the material at the proximal end 425 and/or the distal end 427 may be more flexible than the material at the midpoint 428.

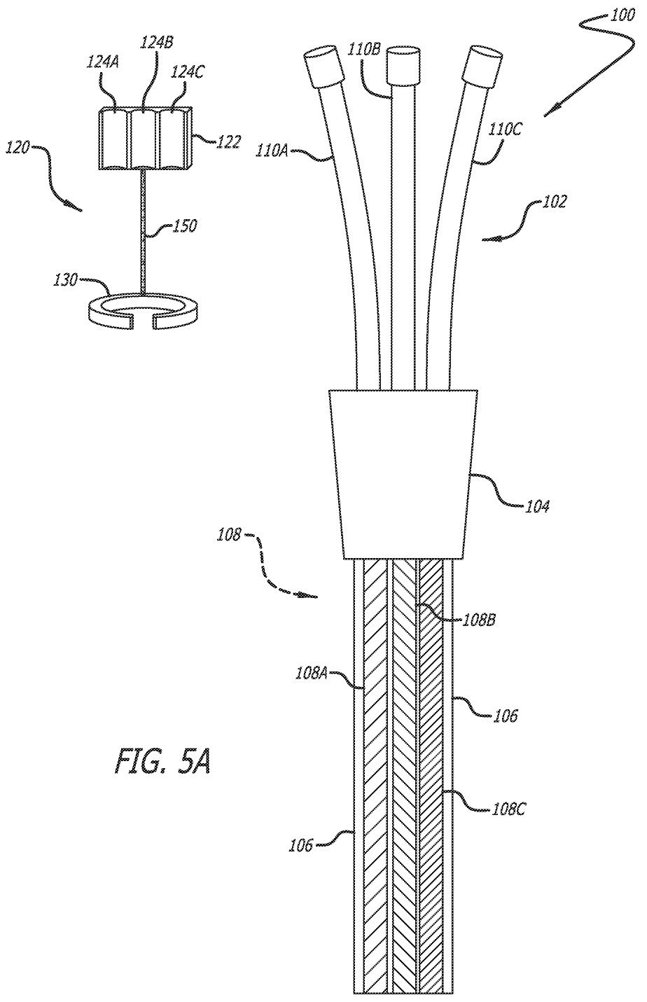

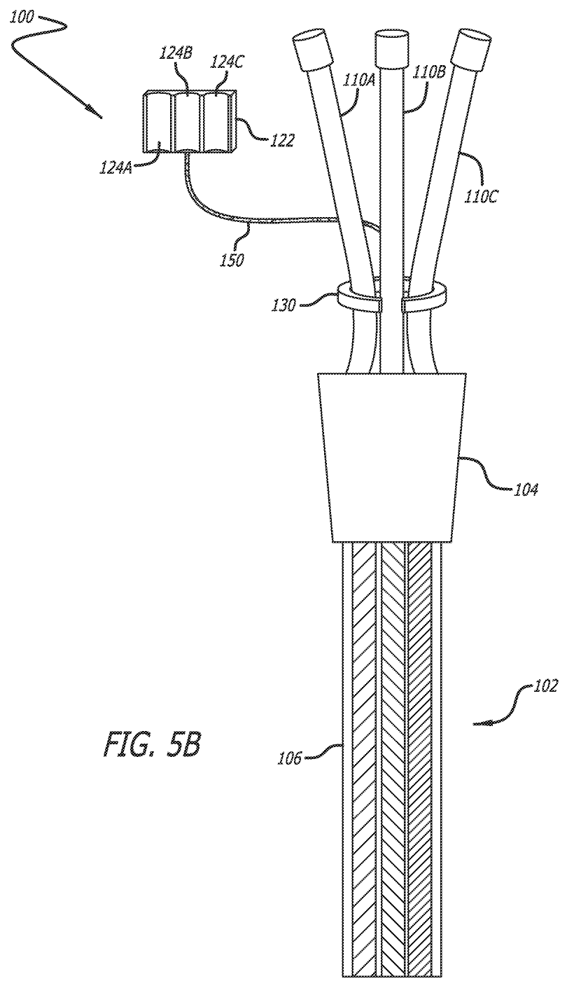

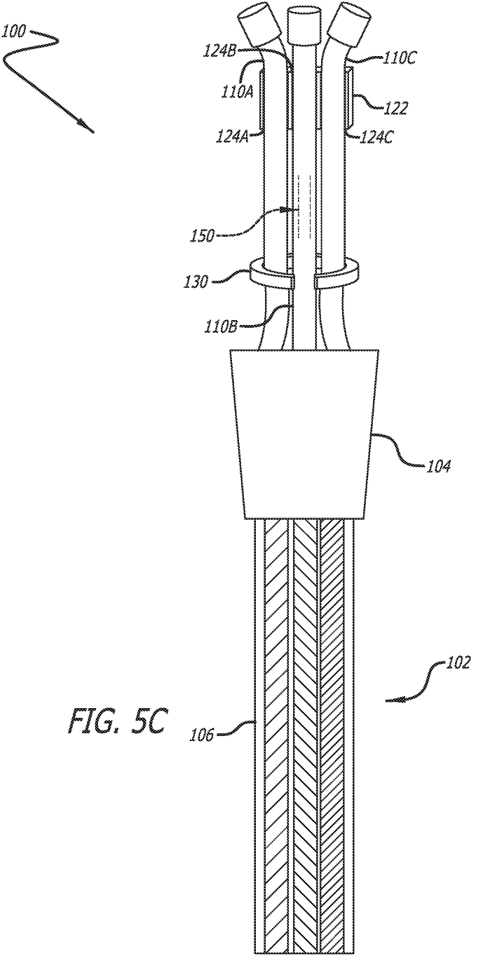

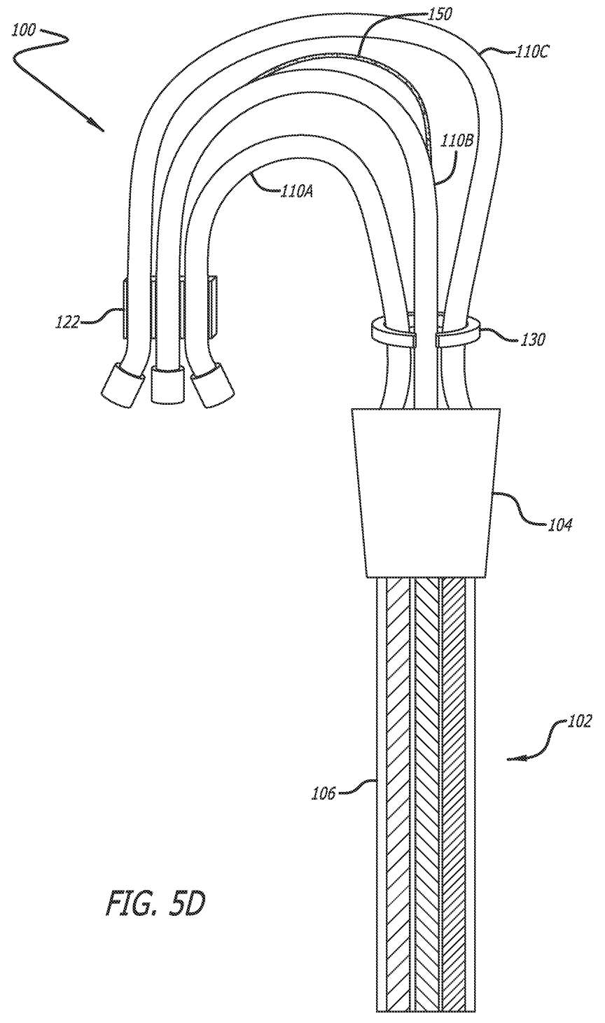

FIGS. 5A-5D illustrate a various perspective views of the catheter assembly 100 depicting an exemplary method of securing and organizing the extension legs 110 of the catheter 102. FIG. 5A illustrates the catheter assembly 100 in a decoupled state, i.e., where the extension leg securement apparatus 120 is separated from the catheter 102. The catheter 102 includes the hub 104 coupled between the catheter tube 106 and (i) a first extension leg 110A, in fluid communication with a first lumen 108A, (ii) a second extension leg 110B in fluid communication with a second lumen 108B, and (iii) a third extension leg 110C in fluid communication with a third lumen 108C. The extension leg securement apparatus 120 includes extension leg ring 130 and the extension leg organizer 122 having a first channel 124A, a second channel 124B, and a third channel 124C.

FIG. 5B illustrates the catheter assembly 100 in a partially coupled state. The extension leg ring 130 is coupled with extension legs 110A-110C. In some instances, the clinician individually insert each of the extension legs 110A-110C through the opening 118 of the extension leg ring 130 so that the extension leg ring 130 constrains the location of the extension legs 110A-110C as a group. In some instances, the extension leg ring 130 may be used to constrain the extension legs 110A-110C adjacent the hub 104.

FIG. 5C illustrates the catheter assembly 100 in a fully coupled state. The extension leg organizer 122 is individually coupled to the extension legs 110A-110C at a location proximal the extension leg ring 130. In some embodiments, the first channel 124A may be coupled to the first extension leg 110A, the second channel 124B may be coupled to the second extension leg 110B, and the third channel 124C may be coupled to the third extension leg 110C. In some embodiments, the clinician may couple extension legs 110A- 110C to the extension leg organizer 122 after coupling extension leg ring 130 to the extension legs 110A-110C. In some embodiments, the clinician may couple the extension legs 110A-110C the extension leg organizer 122 and/or the extension leg ring 130 prior to inserting the catheter 102 into the patient.

FIG. 5D illustrates the catheter assembly 100 in the fully coupled state and the extension leg securement apparatus 120 is disposed in a bended shape. More specifically, the extension leg organizer 122 and the extension leg ring 130 are coupled to the extension legs 110A-110C, and the bendable wire 150 is bent by the clinician away from the straight shape of FIGS. 5A-5C into a curved shape. In some embodiments, bending the extension leg organizer 122 away from the extension leg ring 130 may locate the extension legs 110A-110B so as to be easily accessible by the clinician. It can be appreciated that these steps may be performed in any order. For example, the bendable wire 150 may be bent to desired configuration before the extension legs 110A-110C are coupled to the channels 124A-124C, and/or the extension leg organizer 122 may be coupled to the extension legs 110A-110C before the extension leg ring 130 is coupled to the extension legs 110A-110C.

A method of using a catheter may include all or any subset of the following steps or processes, according to some embodiments. The method may include coupling two or more extension legs of the catheter with an extension leg organizer, such as inserting the extension legs into two or more channels of the extension leg organizer. The method may further include disposing the two or more extension legs within the extension leg ring, where the extension leg ring is coupled with the extension leg organizer.

In some embodiments of the method, the extension leg organizer may be coupled with the extension leg ring via the bendable wire. As such, the method further may further include bending the bendable wire to define a position and an orientation of the extension leg organizer with respect to the extension leg ring.

In some embodiments of the method, the extension leg ring may include a slot configured to individually receive the two or more extension legs therethrough, where the slot defines may define width that is less than a diameter of each of the two or more extension legs. According to such embodiments, disposing the two or more extension legs within an extension leg ring includes displacing the two or more extension legs through the slot.

In some embodiments of the method, the extension leg ring may include a first arm extending along a first partial circumference of the extension leg ring and a second arm extending along a second partial circumference of the extension leg ring. In such embodiments, the second partial circumference is disposed opposite the first partial circumference. In such embodiments, at least one of the first arm or the second arm is deflectable so as to increase the width of the slot. As such, displacing the two or more extension legs through the slot includes deflecting the at least one of the first arm or the second arm.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

What is claimed is:

1. A catheter assembly, comprising:
a catheter having two or more extension legs, wherein each extension leg is in fluid communication with at least one lumen extending along a catheter tube; and
an extension leg securement apparatus attached to each of the two or more extension legs, the extension leg securement apparatus including an extension leg organizer having two or more channels configured to individually receive the two or more extension legs therein.

2. The assembly according to claim 1, wherein each channel is coupled with one of the two or more extension legs via a press fit, a snap fit, or an interference fit.

3. The assembly according to claim 1, wherein each channel is configured to inhibit longitudinal displacement of the one of the two or more extension legs coupled therewith.

4. The assembly according to claim 1, wherein the extension leg organizer is configured to:
be bent by a clinician during use into a curved shape, and
maintain the curved shape thereafter.

5. The assembly according to claim 1, wherein the extension leg securement apparatus further includes an extension leg ring coupled with the extension leg organizer, the extension leg ring extending around the two or more extension legs as a group.

6. The assembly according to claim 5, wherein the extension leg ring is disposed distal the extension leg organizer.

7. The assembly according to claim 5, wherein the extension leg ring includes a slot configured to individually receive the two or more extension legs therethrough.

8. The assembly according to claim 7, wherein the slot defines a width that is less than a diameter of the each of the two or more extension legs.

9. The assembly according to claim 8, wherein:
the extension leg ring includes a first arm extending along a first partial circumference of the extension leg ring and a second arm extending along a second partial circumference of the extension leg ring, the second partial circumference disposed opposite the first partial circumference, and
at least one of the first arm or the second arm is deflectable so as to increase the width of the slot.

10. The assembly according to claim 5, wherein the extension leg securement apparatus further includes a bendable wire coupled between the extension leg ring and the extension leg organizer.

11. The assembly according to claim 10, wherein the bendable wire is configured to:
be bent by a clinician during use to define a position and an orientation of the extension leg organizer with respect to the extension leg ring, and
maintain the position and the orientation of the extension leg organizer with respect to the extension leg ring.

12. The assembly according to claim 1, wherein the extension leg securement apparatus further includes a second extension leg organizer coupled with the extension leg organizer, the second extension leg organizer having two or more channels configured to individually receive the two or more extension legs therein.

13. The assembly according to claim 12, wherein the extension leg securement apparatus further includes a bendable wire coupled between the second extension leg organizer and the extension leg organizer.

14. The assembly according to claim 13, wherein the bendable wire is configured to:
be bent by a clinician during use to define a position and an orientation of the extension leg organizer with respect to the second extension leg organizer, and
maintain the position and the orientation of the extension leg organizer with respect to the second extension leg organizer.

15. A catheter extension leg securement apparatus, comprising:
an extension leg organizer having two or more channels configured to individually receive two or more extension legs of a catheter therein.

16. The apparatus according to claim 15, wherein each channel is configured to couple with one of the two or more extension legs via a press fit, a snap fit, or an interference fit.

17. The apparatus according to claim 15, wherein each channel is configured to inhibit longitudinal displacement of the one of the two or more extension legs coupled therewith.

18. The apparatus according to claim 15, wherein the extension leg organizer is configured to:
be bent by a clinician during use into a curved shape, and
maintain the curved shape thereafter.

19. The apparatus according to claim 15, further comprising:
an extension leg ring coupled with the extension leg organizer, the extension leg ring configured to extend around the two or more extension legs as a group.

20. The apparatus according to claim 19, wherein the extension leg ring includes a slot configured to individually receive the two or more extension legs therethrough.

21. The apparatus according to claim 20, wherein the slot defines a width that is less than a diameter of each of the two or more extension legs.

22. The apparatus according to claim 21, wherein:
the extension leg ring includes a first arm extending along a first partial circumference of the extension leg ring and a second arm extending along a second partial circumference of the extension leg ring, the second partial circumference disposed opposite the first partial circumference, and
at least one of the first arm or the second arm is deflectable so as to increase the width of the slot.

23. The apparatus according to claim 19, further comprising:
a bendable wire coupled between the extension leg ring and the extension leg organizer.

24. The apparatus according to claim 23, wherein the bendable wire is configured to:
be bent by a clinician during use to define a position and an orientation of the extension leg organizer with respect to the extension leg ring, and
maintain the position and the orientation of the extension leg organizer with respect to the extension leg ring.

25. The apparatus according to claim 15, further comprising:
a second extension leg organizer coupled with the extension leg organizer, the second extension leg organizer having two or more channels configured to individually receive the two or more extension legs therein.

26. The apparatus according to claim 25, further comprising:
a bendable wire coupled between the second extension leg organizer and the extension leg organizer.

27. The apparatus according to claim 26, wherein the bendable wire is configured to:
be bent by a clinician during use to define a position and an orientation of the extension leg organizer with respect to the second extension leg organizer, and
maintain the position and the orientation of the extension leg organizer with respect to the second extension leg organizer.

28. A method of using a catheter, comprising:
coupling two or more extension legs of the catheter with extension leg organizer, the extension leg organizer including two or more channels configured to receive the two or more extension legs therein; and
disposing the two or more extension legs within an extension leg ring coupled with the extension leg organizer.

29. The method according to claim 28, wherein the extension leg organizer is coupled with the extension leg ring via a bendable wire, the method further comprising bending the bendable wire to define a position and an orientation of the extension leg organizer with respect to the extension leg ring.

30. The method according to claim 28, wherein:
the extension leg ring includes a slot configured to individually receive the two or more extension legs therethrough,
the slot defines a width that is less than a diameter of each of the two or more extension legs, and
disposing the two or more extension legs within an extension leg ring includes displacing the two or more extension legs through the slot.

31. The method according to claim 30, wherein:
the extension leg ring includes a first arm extending along a first partial circumference of the extension leg ring and a second arm extending along a second partial circumference disposed opposite the first partial circumference,
at least one of the first arm or the second arm is deflectable so as to increase the width of the slot,
displacing the two or more extension legs through the slot includes deflecting the at least one of the first arm or the second arm.
